(19) United States
(12) Patent Application Publication
(10) Pub. No.: US 2011/0177297 A1
(43) Pub. Date: Jul. 21, 2011

(54) METHOD OF MANUFACTURING SOLID MICROSTRUCTURE AND SOLID MICROSTRUCTURE MANUFACTURED BASED ON SAME

(75) Inventors: Hyungil Jung, Seoul (KR); JungDong Kim, Seoul (KR); Kwang Lee, Seoul (KR)

(73) Assignee: NURIM WELLNESS CO. LTD., Seoul (KR)

(21) Appl. No.: 13/122,401

(22) PCT Filed: Oct. 1, 2009
(86) PCT No.: PCT/KR2009/005653

§ 371 Date: Apr. 1, 2011

(30) Foreign Application Priority Data
Oct. 2, 2008 (KR) 10-2008-0097111

(51) Int. Cl. B32B 3/26; A61M 5/158

(52) U.S. Cl. 428/172; 427/2.28

(57) ABSTRACT

There are provided a method of forming a solid microstructure, comprising: (a) preparing a viscous composition on a substrate; (b) contacting a contact protrusion of a lifting support with the viscous composition; (c) blowing air on the viscous composition to condensate and solidify the viscous composition; and (d) cutting the resultant material of step (c) to form the microstructure, and a solid microstructure prepared thereby. A solid microstructure may be manufactured, which can easily include drugs without denaturation or deactivation, has microscale diameters and sufficient effective length and hardness and is also sensitive to heat, and a solid microstructure with desired characteristics (for example, effective length, top diameter and hardness) may also be easily and quickly manufactured at a lower production cost.

### BACKGROUND

1. Field of the Invention

The present invention relates to a method of manufacturing a solid microstructure using an air blowing technique and a solid microstructure manufactured using the same.

2. Discussion of Related Art

While various drugs and therapeutic agents for treating diseases have been developed, in delivery into a human body, they still have aspects in need of improvement such as transmitting across biological barriers (e.g., skin, oral mucosa, blood-brain barriers, etc.) and efficiency of drug delivery.

Generally, drugs are orally administered in tablet or capsule formulation. However, since various drugs are digested or absorbed in the gastrointestinal tract or lost due to mechanisms occurring in the liver, these drugs cannot be effectively delivered through only such oral administration. In addition, some drugs cannot be effectively diffused through an intestinal mucosa. Furthermore, drug compliance in patients becomes a problem (for example, in patients who need to take drugs at predetermined intervals, or critical patients who cannot take drugs).

Another common technique for delivering drugs and phytonutrients is using a conventional needle. This technique is more effective than oral administration, but may cause pain in an injected area, local damage to the skin, bleeding, and infection in the injected area.

To solve these problems, various microstructures including a microneedle have been developed.

The microneedle currently developed has been used to deliver drugs to a human body, collect blood, and detect analytes from the human body.

The microneedle is characterized by piercing the skin without pain and external injury as compared with a conventional needle. For the painless piercing of the skin, the diameter of a tip of the microneedle for the minimum needle sharpness is an important factor. In addition, since the microneedle needs to pierce the 10 to 20 μm-thick stratum corneum, which is the strongest barrier of skin, the microneedle needs to have sufficient physical hardness. Furthermore, a reasonable length of the microneedle to deliver drugs to capillaries should also be considered so as to increase the efficiency of drug delivery.

Conventionally, since the proposal of an in-plane type microneedle (“Silicon-processed Microneedles,” Journal of Microelectrochemical Systems 8, 1999), various types of microneedles have been developed. According to the method of manufacturing an out-of-plane type solid microneedle using an etching technique (disclosed in U.S. Patent Publication No. 2002138049, entitled “Microneedle Devices and Methods of Manufacture and Use Thereof”), a solid silicon microneedle was manufactured to have a diameter of 50 to 100 μm and a length of 500 μm. However, the microneedle could not pierce the skin without pain, and had difficulty in delivery of drugs and cosmetic ingredients to a target region.

Meanwhile, Prausnitz (Georgia Institute of Technology, U.S.A.) has suggested a method of manufacturing a biodegradable polymer microneedle by producing a template by performing etching or photolithography on glass (Biodegradable Polymer Microneedles: Fabrication, Mechanics and Transdermal Drug Delivery, Journal of Controlled Release 104, 2005, 5166). Further, in 2006, a method of manufacturing a biodegradable solid microneedle by loading a material manufactured in a capsule type at an end of a template manufactured by photolithography was suggested (Polymer Microneedles for Controlled-Release Drug Delivery, Pharmaceutical Research 23, 2006, 1008). According to the above-mentioned method, it is easy to load a drug which can be manufactured in a capsule type, but when a large amount of such a drug is loaded, the microneedle is degraded in hardness, and thus there is a limit to the application to a drug that needs to be administered in a large dose.

In 2005, an absorbable microneedle was manufactured by Nano Device and Systems Inc. (Japanese Patent Publication No. 2005154321; and “Sugar Micro Needles as Transdermic Drug Delivery System,” Biomedical Microdevices 7, 2005, 185). Such an absorbable microneedle is used in drug delivery or cosmetics without removal of the microneedle inserted intradermally. According to the above-mentioned method, a composition prepared by mixing maltose and a drug was applied to a template and then solidified so as to manufacture a microneedle. The Japanese patent discloses that an absorbable microneedle for transdermal absorption of drugs is manufactured. However, the transdermal delivery of the drugs was accompanied by pain. In addition, due to a technical limit in the manufacture of a template, it was impossible to manufacture a microneedle whose tip had a suitable diameter to achieve painless absorption and which had a length required for effective drug delivery, that is, a length of 1 mm or more.

A biodegradable microneedle suggested by Prausnitz (Georgia Institute of Technology, U.S.A.) in 2008 was manufactured using a polydimethylsiloxane (PDMS) template and a material prepared by mixing polyvinylpyrrolidone (PVP) and methacrylic acid (MAA) (Minimally Invasive Protein Delivery with Rapidly Dissolving Polymer Microneedles, Advanced Materials 2008, 1). Further, a microneedle was manufactured by injecting carboxymethylcellulose into a pyramid-structure template (Dissolving Microneedles for Transdermal Drug Delivery, Biomaterials 2007, 1). However, the method using a template has a limit in that, although a microneedle may be manufactured in a rapid and simple way, the microneedle cannot be manufactured while adjusting a diameter and length thereof.

Meanwhile, the skin is composed of a stratum corneum (<20 μm), an epidermis (<100 μm), and a dermis (300 to 2,500 μm), which are sequentially stacked from the outer layer of the skin. Accordingly, to deliver drugs and phytonutrients to a specific layer of the skin without pain, the microneedle needs to be manufactured to have a top diameter of approximately 30 μm, an effective length of 200 to 2,000 μm, and a sufficient hardness to pierce the skin, which is also effective in delivery of the drugs and phytonutrients. In addition, to deliver drugs and phytonutrients by a biodegradable solid microneedle, it is necessary to exclude processes, which may destroy the activities of the drugs and phytonutrients, such as high heat treatment, treatment with an organic solvent, etc. from the process of manufacturing a microneedle.

Conventional solid microneedles are manufactured using limited materials such as silicon, polymers, metal, glass, etc. due to the limit of the manufacturing method, and the tip is manufactured to have a diameter of 50 to 100 μm and a length of 500 μm, thereby making it difficult to accomplish the desired effects. Accordingly, there are ongoing demands for a method of manufacturing a microneedle which has an enough thin diameter to pierce the skin without pain, a sufficient length to deeply penetrate into the skin, and a sufficient hardness without a particular limit to a material.

Throughout the specification, numerous papers and patents are referenced and their citations are noticed. The level in the art to which the present invention pertains and the sprit and scope of the present invention will be more apparent from the papers and patents, the disclosures of which are incorporated herein by reference in its entirety.

### SUMMARY OF THE INVENTION

The inventors have made efforts to develop a solid microstructure having a micro-sized diameter, and a sufficient effective length and hardness, and capable of easily containing heat-sensitive drugs without denaturalization or inactivation. As a result, they had confirmed that the solid microstructure manufactured through a process including the steps of contacting, lifting, blowing, condensation and solidification, without heat treatment, has these characteristics. Therefore, the present invention is completed based on the above facts.

Accordingly, the present invention is directed to a method of manufacturing a solid microstructure.

The present invention is also directed to a solid microstructure.

The foregoing and other objects, features, and advantages of the invention will be more apparent from the more particular description of exemplary embodiments of the invention as illustrated in the accompanying drawings.

According to an aspect of the present invention, a method of manufacturing a microstructure according to the present invention includes:
(a) preparing a viscous composition on a substrate;
(b) contacting a contact protrusion of a lifting support with the viscous composition;
(c) blowing air on the viscous composition to condensate and solidify the viscous composition; and
(d) cutting the resultant material of step (c) to form the microstructure.

According to another aspect of the present invention, a solid microstructure manufactured by the method of the present invention is provided.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 illustrates a lifting structure used to manufacture a microstructure in accordance with an exemplary embodiment of the present invention;

FIG. 2 is a schematic view showing a process of manufacturing the microstructure in accordance with an exemplary embodiment of the present invention; and

FIG. 3 shows a patterned microstructure formed on a substrate according to the method in accordance with the present invention, in which FIG. 3A is a plan view of the microstructure, FIG. 3B is a front view of the microstructure, and FIG. 3C is a perspective view of the microstructure.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various changes and modifications can be made without departing from the scope of the invention.

The inventors have made efforts to develop a solid microstructure having a micro-sized diameter, and a sufficient effective length and hardness, and capable of easily containing heat-sensitive drugs without denaturalization or inactivation. As a result, the inventors have developed a novel method of manufacturing a solid microstructure, which includes lifting viscous materials, blowing air on the materials and condensing the materials, and has the above advantages. Accordingly, they have confirmed that the solid microstructure having desired characteristics (for example, an effective length, and a diameter and hardness of a tip) can be more simply and rapidly manufactured at a low cost.

A method of the present invention will be described according to the respective steps in detail.

Step (a): Preparing a Viscous Composition on a Substrate

A material used in the present invention to manufacture a microstructure is a viscous composition. In this specification, the term “viscous composition” means a composition capable of forming a microstructure when lifted while contacting a lifting support used in the present invention.

Viscosity of the viscous composition may be variously varied according to a kind, concentration, and temperature of a material included in the composition, addition of a viscosity modifying agent, or the like, and appropriately adjusted according to the objects of the present invention. Viscosity of the viscous composition may be adjusted by inherent viscosity of a viscous material, or may be adjusted using an additional viscosity modifying agent.

For example, viscosity of the composition may be appropriately adjusted by adding a viscosity modifying agent conventionally used in the art, for example, hyaluronic acid and salts thereof, polyvinyl pyrrolidone, cellulose polymer, dextran, gelatin, glycerin, polyethylene glycol, polysorbate, propylene glycol, povidone, carbomer, gum ghatti, guar gum, glucomannan, glucosamine, dammer resin, rennet casein, locust bean gum, microfibillated cellulose, psyllium seed gum, xanthan gum, arabino galactan, gum arabic, alginic acid, gellan gum, carrageenan, karaya gum, curdlan, chitosan, chitin, tara gum, tamarind gum, tragacanth gum, furcelleran, pectin, or pullulan, to a composition including a major element of a solid microstructure, for example, a biocompatible material. Preferably, the viscosity of the viscous composition used in the present invention is 200,000 cSt or less.

According to an exemplary implementation of the present invention, the viscous composition used in the present invention includes a biocompatible or biodegradable material. Here, the term “biocompatible material” refers to a material which is substantially non-toxic to the human body, chemically inactive, and devoid of immunogenicity. The term “biodegradable material” refers to a material which can be degraded by a body fluid, microorganisms, or the like.

Preferably, the viscous composition used in the present invention include hyaluronic acid and salts thereof, polyvinylpyrrolidone, cellulose polymer, dextran, glycerin, polyethyleneglycol, polysorbate, propyleneglycol, povidone, carbomer, gum ghatti, guar gum, glucomannan, glucosamine, dammer resin, rennet casein, locust bean gum, microfibrillated cellulose, psyllium seed gum, xanthan gum, arabino galactan, gum arabic, alginic acid, gelatin, gellan gum, carrageenan, karaya gum, curdlan, chitosan, chitin, tara gum, tamarind gum, tragacanth gum, furcelleran, pectin, or pullulan. More preferably, the viscous material included in the viscous composition used in the present invention is cellulose polymer, more preferably, hydroxypropyl methylcellulose, hydroxyalkyl cellulose (preferably, hydroxyethyl cellulose or hydroxypropyl cellulose), ethyl hydroxyethyl cellulose, alkylcellulose, and carboxymethylcellulose, still more preferably, hydroxypropyl methylcellulo se or carboxymethylcellulose, and most preferably, carboxymethylcellulose.

Alternatively, the viscous composition may include a biocompatible and/or biodegradable material as a major element.

The biocompatible and/or biodegradable material, which may be used in the present invention, is, for example, polyester, polyhydroxyalkanoates (PHAs), poly(α-hydroxy acid), poly(β-hydroxy acid), poly(3-hydroxybutirate-co-velerate) (PHBV), poly(3-hydroxyproprionate) (PHP), poly(3-hydroxyhexanoate) (PHH), poly(4-hydroxy acid), poly(4-hydroxybutirate), poly(4-hydroxyvalerate), poly(4-hydroxyhexanoate), poly(esteramide), polycaprolactone, polylactide, polyglycolide, poly(lactide-co-glycolide) (PLGA), polydioxanone, polyortoester, polyetherester, polyanhydride, poly(glycolic acid-co-trimethylene carbonate), polyphosphorester, polyphosphorester urethane, poly(amono acid), polycyanoacrylate, poly(trimethylene carbonate), poly(iminocarbonate), poly(tyrosine carbonate), polycarbonate, poly(tyrosine arylate), polyaklylene oxalate, polyphosphazenes, PHA-PEG, ethylene vinyl alcohol copolymer (EVOH), polyurethane, silicon, polyester, polyolefin, polyisobutylene and ethylene-alphaolefin copolymer, styrene-isobutylene-styrene triblock copolymer, acryl polymer and copolymer, vinyl halide polymer and copolymer, polyvinyl chloride, polyvinyl ether, polyvinyl methyl ether, polyvinylidene halide, polyvinylidene fluoride, polyvinylidene chloride, polyfluoroalkene, polyperfluoroalkene, polyacrylonitrile, polyvinyl ketone, polyvinyl aromatics, polystyrene, polyvinyl ester, polyvinyl acetate, ethylene-methyl, methacrylate copolymer, acrylonitrile-styrene copolymer, ABS resin and ethylene-vinyl acetate copolymer, polyamide, alkyd resin, polyoxymethylene, polyimide, polyether, polyacrylate, polymethacrylate, polyacrylic acid-co-maleic acid, chitosan, dextran, cellulose, heparin, hyaluronic acid, alginate, inulin, starch, or glycogen, preferably, polyester, PHAs, poly(α-hydroxy acid), poly((3-hydroxy acid), PHBV, PHP, PHH, poly(4-hydroxy acid), poly(4-hydroxybutirate), poly(4-hydroxyvalerate), poly(4-hydroxyhexanoate), poly(esteramide), polycaprolactone, polylactide, polyglycolide, PLGA, polydioxanone, polyortoester, polyetherester, polyanhydride, poly(glycolic acid-co-trimethylene carbonate), polyphosphorester, polyphosphorester urethane, poly(amono acid), polycyanoacrylate, poly(trimethylene carbonate), poly(iminocarbonate), poly(tyrosine carbonate), polycarbonate, poly(tyrosine arylate), polyaklylene oxalate, polyphosphazenes, PHA-PEG, chitosan, dextran, cellulose, heparin, hyaluronic acid, alginate, inulin, starch, or glycogen.

According to an exemplary implementation of the present invention, the viscous composition used in the present invention is dissolved in an appropriate solvent to show viscosity. Meanwhile, among materials showing viscosity, there are materials that can show viscosity when they are melted by heat. In order to maximize an advantage of a non-heating process, which is one of the advantages of the present invention, the material used in the viscous composition preferably shows viscosity when the material is dissolved in an appropriate material.

A solvent used to dissolve the above-mentioned viscous material to prepare a viscous composition may be, but is not particularly limited to, water, an anhydrous or hydrous low alcohol having 1 to 4 carbon atoms, acetone, ethyl acetate, chloroform, 1,3-butyleneglycol, hexane, diethylether, or butylacetate, preferably water or low alcohol, and more preferably water.

A substrate for accommodating a viscous composition is not particularly limited, but may be manufactured using materials such as a polymer, an organic chemical, a metal, a ceramic, a semiconductor, and so on.

According to an exemplary implementation of the present invention, the viscous composition further includes a drug. A major use of a microstructure of the present invention is as a microneedle, which is used for dermal administration. Accordingly, during preparation of the viscous composition, drugs are mixed with a biocompatible material.

The drugs that may be used in the present invention are not particularly limited. For example, the drugs include chemical drugs, protein medicines, peptide medicines, nucleic acid molecules for gene therapy, nano particles, and so on.

The drugs that may be used in the present invention may include, but are not limited to, for example, antiinflammatory drugs, pain killers, antiarthritics, sedatives, anti-depressants, antipsychotic drugs, nervous sedatives, antianxiety drugs, narcotic antagonists, anti-Parkinson's disease drugs, cholinergic agonists, anticancer drugs, anti-angiogenic drugs, immunosuppressive drugs, anti-virus drugs, antibiotics, appetite suppressants, anticholinergics, antihistaminic agents, anti-migraine agents, hormone drugs, vasodilators for coronary vessels, cerebrovascular vessels or peripheral blood vessels, contraceptive pills, antithrombotics, diuretics, antihypertensives, cardiovascular disease medicines, cosmetic ingredients (e.g., a wrinkle inhibitor, a skin aging inhibitor, or skin whitener), and so on.

According to the exemplary implementation of the present invention, a process of manufacturing the microstructure of the present invention is performed through non-heating treatment. Accordingly, even when the drugs used in the present invention, for example, protein medicines, peptide medicines, nucleic acid molecules for gene therapy and so on, are sensitive to heat, the manufacture of the microstructure including the drugs is possible according to the present invention.

According to the exemplary implementation of the present invention, the method of the present invention is used to manufacture the microstructure including heat-sensitive drugs, and more preferably, protein medicines, peptide medicines, or vitamins (preferably, vitamin C).

The protein/peptide medicines included in the microstructure by the method of the present invention are not particularly limited but may include hormones, hormone analogues, enzymes, enzyme inhibitors, signaling proteins or portions thereof, antibodies or portions thereof, single-chain antibodies, binding proteins or binding domains thereof, antigens, adhesion proteins, structural proteins, regulatory proteins, toxoproteins, cytokine, transcriptional regulatory factors, blood coagulation factors, vaccines, and so on. More specifically, the protein/peptide medicines include insulin, an insulin-like growth factor 1 (IGF-1), growth hormone, erythropoietin, granulocyte-colony stimulating factors (G-CSFs), granulocyte/macrophage-colony stimulating factors (GM-CSFs), interferon alpha, interferon beta, interferon gamma, interleukin-1 alpha and beta, interleukin-3, interleukin-4, interleukin-6, interleukin-2, epidermal growth factors (EGFs), calcitonin, adrenocorticotropic hormone (ACTH), a tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclosporine, exedine, lanreotide, luteinizing hormone-releasing hormone (LHRH), nafarelin, parathormone, pramlintide, T-20 (enfuvirtide), thymalfasin, and ziconotide.

According to the exemplary implementation of the present invention, the viscous composition further includes energy. In this case, the microstructure may be used to transmit or deliver energy such as thermal energy, light energy, electrical energy, and so on. For example, in photodynamic therapy, the microstructure may be used to induce light in a specific part of a human body such that the light can be applied to tissues or intermediates such as light-sensitive molecules.

The substrate for accommodating the viscous composition is not limited but may be formed of materials such as polymers, organic chemicals, metals, ceramics, semiconductors, and so on.

Step (b): Contacting a Contact Protrusion of a Lifting Support with the Viscous Composition

Next, a contact protrusion of a lifting support is in contact with the viscous composition. In order to manufacture the microstructure using viscosity, which is a characteristic of the viscous composition, first, the lifting support must be lowered to contact the contact protrusion with the viscous composition.

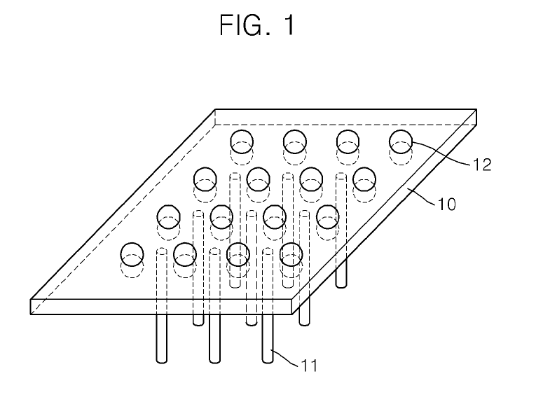

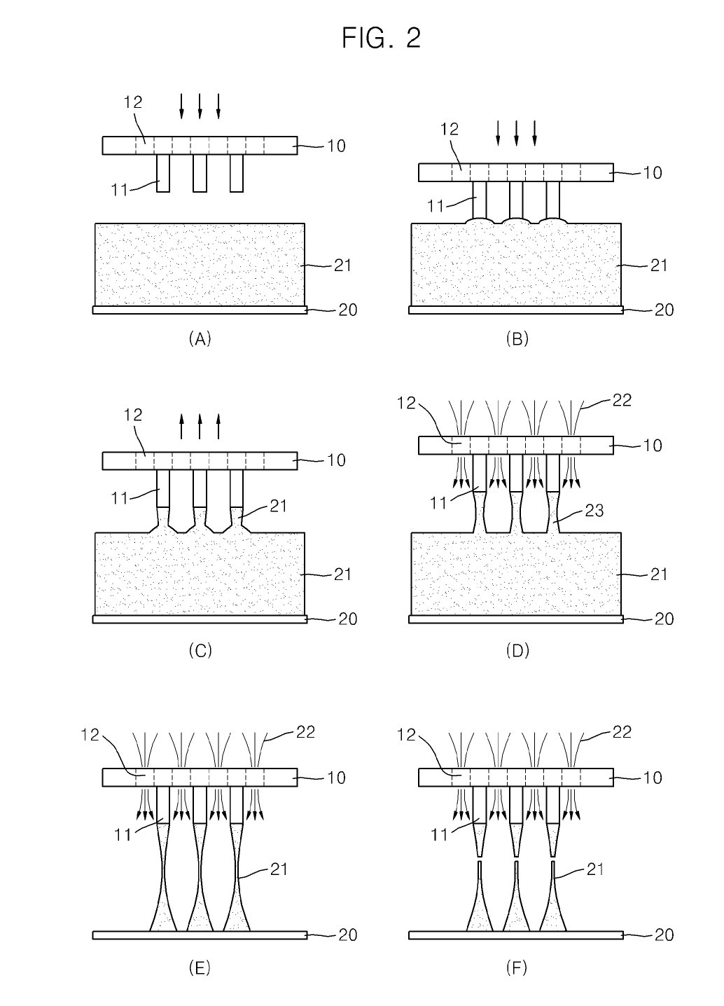

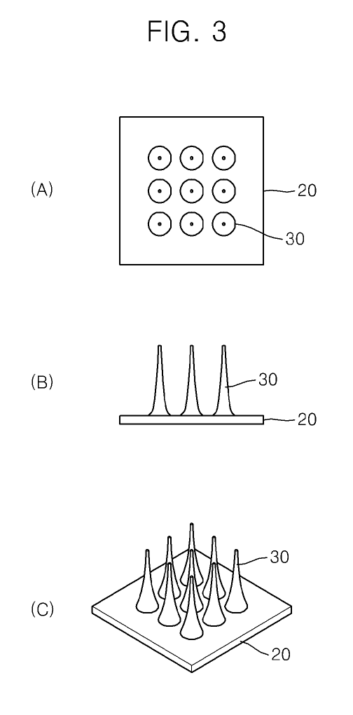

FIG. 1 shows a specific embodiment of the lifting support. The lifting support includes one or more contact protrusions, and, for example, the viscous composition including the biocompatible material is attached to the contact protrusion (see FIG. 2B). According to the exemplary implementation of the present invention, the contact protrusion of the lifting support is patterned (see FIGS. 1 and 2). The patterning is advantageous when the microstructure of the present invention is fabricated in a patch type, and may be fabricated in an array shape including various drugs at the respective microstructures or some of the microstructures (see FIG. 3).

According to the exemplary implementation of the present invention, air is blown after the viscous composition is in contact with the contact protrusion of the lifting support so that the viscous composition adhered to the contact protrusion is easily condensed to generate the microstructure from the contact protrusion to the substrate (see step (c)). The blowing may be performed by various methods. Most preferably, the blowing is performed through one or more blowing holes formed in the lifting support. When the air is blown on the viscous composition through the blowing hole, a volume of the viscous composition is reduced from a periphery of the viscous composition attached to the contact protrusion such that the microstructure is formed from the contact protrusion.

Step (c): Condensation and Solidification of the Viscous Composition by the Blowing

One of the most important characteristics of the present invention is blowing air on the viscous composition to condense and solidify the viscous composition, thus manufacturing the microstructure. In this specification, the term “condensation” means that a volume of a viscous material is reduced in comparison with an initial volume during a process of solidifying the viscous material from a fluid state.

In general, in order to manufacture the microstructure, the viscous composition is drawn beyond an effective length of the microstructure. Meanwhile, the present invention provides the resultant microstructure using a condensation property of the viscous composition, and the perfect effective length of the microstructure is formed by the blowing. That is, during a process of condensing and solidifying the viscous composition used to form the microstructure in a state in which the viscous composition is adhered to the lifting support, since a large area of the viscous composition adhered to the contact protrusion is exposed to the blown air and the exposed area is more rapidly solidified than the viscous composition disposed around a lower part of the contact protrusion, the viscous composition disposed at a lower part of the intermediate structure is concentrated on the intermediate structure to be condensed. Eventually, the microstructure having an effective length and hardness that can penetrate the skin is formed about the intermediate structure (see FIGS. 2D and 2E).

According to the exemplary implementation of the present invention, step (b-2) of lifting the lifting support is further included between steps (b) and (c). While the present invention can manufacture the microstructure without lifting the lifting support, the lifting support may be lifted to variously manufacture the microstructure in a desired shape. In this specification, the term “lifting” means that the microstructure is lifted up using viscosity or adhesion of the viscous composition. According to the exemplary implementation of the present invention, the intermediate structure formed by the lifting has a length smaller than that of the resultant microstructure, more preferably, a length of 1/100 to 80/100 of the length of the resultant microstructure, most preferably, a length of 5/100 to 70/100 of the length of the resultant microstructure.

The reason for manufacturing the microstructure having the effective length even though the lifting support is lifted to a height lower than the length of the resultant microstructure is that the viscous composition is condensed and solidified about the intermediate structure when air is blown to the viscous composition.

The lifting speed and time are not particularly limited. Preferably, the lifting speed may be 1 to 50 μm/s, and more preferably, 3 to 30 μm/s, and the lifting time may be 10 to 600 seconds, more preferably 20 to 300 seconds, and most preferably 30 to 200 seconds.

The condensation and solidification of the viscous composition may be performed through the blowing, which may be performed in various ways. According to the exemplary implementation of the present invention, the blowing is performed through one or more blowing holes formed in the lifting support used in the present invention. Alternatively, the blowing may be directly performed on the viscous composition, not passing through the lifting support, or may be simultaneously performed with blowing through the lifting support. The blowing through the blowing holes of the lifting support is advantageous for the uniform blowing, and is also advantageous in forming the microstructure whose shape is not distorted.

The blowing for manufacturing the microstructure may be induced in various ways. The blowing ways are not particularly limited as long as the lifting characteristics and the condensation and solidification properties of the viscous composition are used. Three typical and exemplary implementations will be described as follows.

According to a first implementation, the blowing is performed simultaneously with the lifting of step (b-2). After completion of the lifting, the air is continuously blown to finally manufacture the microstructure.

According to a second implementation, the blowing is performed after the lifting of step (b-2).

According to a third implementation, the blowing and the lifting of step (b-2) are non-continuously and alternately performed. In this case, the lifting and the blowing are alternately performed through several steps, and until the entire lifting is completed, the lifting and the blowing may be performed through various steps according to characteristics of the viscosity and the solidification speed of the viscous composition. According to the third implementation, the condensation and solidification and the lifting are alternately performed until the entire lifting of the lifting support is completed.

According to the exemplary implementation of the present invention, the blowing is performed through the first or second implementations.

Step (d): Formation of the Resultant Microstructure by Cutting

A portion of the microstructure including the effective length is cut from the resultant matter of step (c) to finally obtain the microstructure. The cutting may be performed in various ways, for example, physical cutting or laser cutting.

The present invention may provide various microstructures, preferably, a microneedle, a microblade, a microknife, a microfiber, a microspike, a microprobe, a microbarb, a microarray, or a microelectrode, more preferably, a microneedle, a microblade, a microknife, a microfiber, a microspike, a microprobe, or a microbarb, and most preferably, a solid microneedle.

According to the exemplary implementation of the present invention, the microstructure of the present invention includes a tip having a diameter of 1 to 500 μm, more preferably 2 to 300 μm, and most preferably 5 to 100 μm, and an effective length of 100 to 10,000 μm, more preferably 200 to 10,000 μm, still more preferably 300 to 8,000 μm, and most preferably 500 to 2,000 μm.

The term “tip” of the microstructure used in the specification means a distal end of the microstructure having a minimal diameter. The term “effective length” used in the present invention means a vertical length from the tip of the microstructure to a surface of the support. The term “solid microneedle” used in the specification means a microneedle integrally formed with the microstructure having a non-hollow structure.

The diameter, length and/or shape of the microstructure may be adjusted by varying the diameter of the contact protrusion of the lifting support, the blowing intensity, or the viscosity of the viscous composition.

Characteristics and advantages of the present invention will be summarized as follows.

(i) The present invention provides the method of manufacturing the solid microstructure through the processes including contacting, blowing, condensation and solidification, without heat treatment, which have not been adapted in a conventional art.

(ii) According to the present invention, it is possible to manufacture the solid microstructure having a micro-sized diameter, a sufficient effective length and hardness, and capable of easily containing heat-sensitive drugs without denaturalization and inactivation.

(iii) According to the present invention, it is possible to simply and rapidly manufacture the solid microstructure having desired characteristics (for example, an effective length, a tip diameter and hardness) at a low production cost.

Hereinafter, the present invention will be described in detail through the following embodiment. It will be appreciated by those skilled in the art that the embodiment is only provided to more specifically describe the present invention and the scope of the present invention is not limited by the embodiment.

### Example

As a viscous composition 21 for manufacturing a microstructure, carboxymethylcellulose (high viscosity, sigma) was used. 0.4 mg of carboxymethylcellulose was dissolved in deionized water to make a 2% (w/v) solution. 2% carboxymethylcellulose was coated on a glass substrate 20, and then, a lifting support 10 having 3×3 contact protrusions with a diameter of 500 μm was in contact therewith (see FIG. 2A). After the contacting with the lifting support, air was blown through blowing holes 12 for 5 minutes to come in strong contact with the contact protrusions while smoothly curing the carboxymethylcellulose (see FIG. 2B). The lifting support was lifted (the entire lifting height: 716.7 μm) for 1 minute at a speed of 11.945 μm/s to form an intermediate structure 23 (see FIG. 2C). Moisture in the carboxymethylcellulose was dried by blowing the air through the blowing holes between the substrate and the lifting support after contact with the lifting support (see FIG. 2D). While the moisture was running dry, the carboxymethylcellulose was cured from the lifting support to the substrate to manufacture the microneedle (see FIG. 2E). The cured solid microneedle was cut using micro-scissors (see FIG. 2F). As a result, the microneedle 30 having a tip diameter of 50 μm and an effective length of 1,200 μm was manufactured (see FIG. 3). At this time, the diameter of the microneedle may be adjusted by varying the diameter of the contact protrusion. In addition, it could be confirmed that the shape of the prepared solid microneedle was changed by varying the blowing intensity of air passed through the blowing holes, or the viscosity of the carboxymethylcellulose. When carboxymethylcellulose (low viscosity, sigma) was used, the microstructure could be manufactured in a microneedle shape as long as the carboxymethylcellulose had a concentration of 10% (w/v), and a needle having a larger diameter could be manufactured.

Further, the microstructure was manufactured using chitosan (low molecular weight, sigma) as the viscous composition 21. After mixing 100 μl of acetic acid with 10 ml of deionized water, 0.46 g of chitosan was dissolved to manufacture a 30% (w/v) viscous chitosan material. After applying 100 μl of the prepared viscous chitosan material onto a prepared substrate 20, the lifting support 10 having 4×4 contact protrusions with a diameter of 400 μm was in contact with the chitosan. While slowly curing the chitosan through blowing for 5 minutes, the contact protrusions and the chitosan were solidified to be strongly adhered to each other. While maintaining the weak blowing, the lifting support was lifted for 30 seconds at a speed of 0.6 mm/min and then lifted for 2 minutes and 30 seconds at a reduced speed 0.2 mm/min to form an intermediate structure 23 (see FIGS. 2B to 2D). When the lifting was completed, the air was blown for approximately 15 to 20 minutes to cure the viscous material, thereby manufacturing the microstructure in a microneedle shape (see FIG. 2E). The cured microstructure was cut using micro-scissors (see FIG. 2F). As a result, it can be seen that the completed microstructure having a tip diameter of 50 μm and an effective length of 800 μm was manufactured (see FIG. 3). At this time, the diameter and length of the microstructure can be adjusted by varying the diameter of the contact protrusion, and the lifting speed and time.

A microstructure was manufactured using hyaluronic acid (sodium salt, sigma) as another viscous composition 21. 0.2 g of hyaluronic acid (low molecular weight, 10,000 to 15,000 MW) and 0.3 g of hyaluronic acid (high molecular weight, 1,000,000 to 1,500,000 MW) were dissolved in 10 μl of deionized water to manufacture a viscous hyaluronic acid composition. After applying 100 μl of the prepared viscous hyaluronic acid material onto a prepared substrate 20, the lifting support 10 having 4×4 contact protrusions with a diameter of 400 μm was in contact with the viscous material. While slowly curing the hyaluronic acid through blowing for 5 minutes, the contact protrusions and the chitosan were solidified to be strongly adhered to each other. While maintaining the weak blowing, the lifting support was lifted for 30 seconds at a speed of 0.6 mm/min and then lifted for 2 minutes and 30 seconds at a reduced speed 0.2 mm/min to form an intermediate structure 23 (see FIGS. 2B to 2D). When the lifting was completed, the air was blown for approximately 15 to 20 minutes to cure the viscous material, thereby manufacturing the microstructure in a microneedle shape (see FIG. 2E). The cured microstructure was cut using micro-scissors (see FIG. 2F). As a result, it can be seen that the completed microstructure having a tip diameter of 40 μm and an effective length of 800 μm was manufactured (see FIG. 3). At this time, the diameter and length of the microstructure can be adjusted by varying the diameter of the contact protrusion, and the lifting speed and time.

A microstructure was manufactured using a viscous composition 21 in which the hyaluronic acid (sodium salt, sigma) and the carboxymethylcellulose (low viscosity, sigma) were mixed. 0.2 g of carboxymethylcellulose and 0.2 g of hyaluronic acid (high molecular weight of 1,000,000 to 1,500,000 MW) were dissolved in 20 μl of deionized water to manufacture a viscous composition. After coating the mixed viscous composition onto a prepared substrate 20, the lifting support 10 having 4×4 contact protrusions with a diameter of 500 μm was in contact with the viscous composition (see FIG. 2A). After contact with the lifting support, while passing the air through the blowing holes 12 to slowly cure the mixed viscous composition for 5 minutes, the contact protrusions and the viscous composition were solidified to be strongly adhered to each other (see FIG. 2B). The lifting support was lifted for 1 minute at a speed of 0.6 mm/min and then lifted for 3 minutes at a reduced speed 0.1 mm/min (the entire lifting height: 900 μm) to form an intermediate structure 23 (see FIG. 2C). While continuously blowing the air 22 through the blowing holes between the lifting support and the substrate after contact with the lifting support, moisture of the viscous composition was dried (see FIG. 2D). As the moisture was dried, the carboxymethylcellulose and the hyaluronic acid were cured from the lifting support and the substrate to manufacture the microstructure in a microneedle shape (see FIG. 2E). The cured microstructure was cut using micro-scissors (see FIG. 2F). As a result, it can be seen that the microneedle 30 having a tip diameter of 50 μm and an effective length of 1,200 μm was manufactured (see FIG. 3). At this time, the diameter and length of the microneedle can be adjusted by varying the diameter of the contact protrusion, and the lifting speed and time.

In order to observe variation in diameter of the microstructure according to the diameter of the contact protrusion, the microstructures were manufactured by adjusting the diameter to 200 μm, 250 μm, 300 μm, 350 μm, 400 μm, 450 μm and 500 μm. Carboxymethylcellulose (low viscosity, sigma) was used as a viscous composition, and 0.3 g of the carboxymethylcellulose was dissolved in deionized water to manufacture the microstructure. After coating the viscous composition to the substrate 20, the lifting support 10 having the contact protrusions with the diameter of 200 to 500 μm was in contact with the viscous composition (see FIG. 2A). After contact with the lifting support, while passing the air through the blowing holes 12 to slowly cure the carboxymethylcellulose for 5 minutes, the contact protrusions and the carboxymethylcellulose were solidified to be strongly adhered to each other (see FIG. 2B). The lifting support was lifted for 1 minute at a speed of 0.6 mm/min and then lifted for 3 minutes at a reduced speed of 0.1 mm/min (the entire lifting height: 900 μm) to form an intermediate structure 23 (see FIG. 2C). While continuously blowing the air 22 through the blowing holes between the lifting support and the substrate after contact with the lifting support, moisture in the carboxymethylcellulose was dried (see FIG. 2D). As the moisture was dried, the carboxymethylcellulose was cured from the lifting support and the substrate to manufacture the microstructure in a microneedle shape (see FIG. 2E). It will be appreciated that, when the diameter of the contact protrusion is 300 μm or more, the diameter of the microstructure is increased in proportion to the size of the contact protrusion.

It will be apparent to those skilled in the art that various changes and modifications can be made to the above-described exemplary embodiments of the present invention without departing from the scope of the invention. Thus, it is intended that the present invention covers all such changes and modifications provided they come within the scope of the appended claims and their equivalents.

What is claimed is:

1. A method of manufacturing a microstructure, comprising:
(a) preparing a viscous composition on a substrate;
(b) contacting a contact protrusion of a lifting support with the viscous composition;
(c) blowing air on the viscous composition to condensate and solidify the viscous composition; and
(d) cutting the resultant material of step (c) to form the microstructure.

2. The method according to claim 1, wherein steps (a) through (d) are performed through non-heating treatment.

3. The method according to claim 1, wherein the viscous composition comprises a biocompatible or biodegradable material.

4. The method according to claim 1, wherein the viscous composition comprises a viscous material selected from the group consisting of hyaluronic acid and salts thereof, polyvinyl pyrrolidone, cellulose polymer, dextran, gelatin, glycerin, polyethylene glycol, polysorbate, propylene glycol, povidone, carbomer, gum ghatti, guar gum, glucomannan, glucosamine, dammer resin, rennet casein, locust bean gum, microfibillated cellulose, psyllium seed gum, xanthan gum, arabino galactan, gum arabic, alginic acid, gellan gum, carrageenan, karaya gum, curdlan, chitosan, chitin, tara gum, tamarind gum, tragacanth gum, furcelleran, pectin, and pullulan.

5. The method according to claim 4, wherein the cellulose polymer is selected from the group consisting of hydroxypropyl methylcellulose, hydroxyethyl cellulose, ethyl hydroxyethyl cellulose, hydroxypropyl cellulose, alkylcellulose, and carboxymethylcellulose.

6. The method according to claim 5, wherein the cellulose polymer is hydroxypropyl methylcellulose or carboxymethylcellulose.

7. The method according to claim 1, wherein the lifting support comprises one or more blowing holes.

8. The method according to claim 7, wherein the blowing is performed through the blowing holes of the lifting support.

9. The method according to claim 1, further comprising lifting the lifting support (b-2) between steps (b) and (c).

10. The method according to claim 9, wherein the lifting support is lifted to a height corresponding to 1/100 to 80/100 of the length of the resultant microstructure.

11. The method according to claim 9, wherein lifting the lifting support is performed simultaneously with the blowing on the viscous composition.

12. The method according to claim 9, wherein blowing of step (c) is performed after completion of lifting of step (b-2).

13. The method according to claim 9, wherein step (b-2) and step (c) are non-continuously and alternately performed.

14. The method according to claim 1, wherein the microstructure is a microneedle, a microspike, a microblade, a microknife, a microfiber, a microprobe, a microbarb, a microarray, or a microelectrode.

15. The method according to claim 14, wherein the microstructure is a microneedle.

16. The method according to claim 1, wherein the viscous composition further comprises a drug or energy.

17. A microstructure manufactured through the manufacturing method according to claim 1.
